(19) United States
(12) Patent Application Publication
(10) Pub. No.: US 2008/0166427 A1
(43) Pub. Date: Jul. 10, 2008

(54) Method for Reducing Gastrointestinal Toxicity Due to the Administration of Tegafur

(75) Inventors: Naruo Nomura, Tokushima (JP); Keiko Nomura, Kagawa (JP); Takeshi Tahara, Kanagawa (JP); Hiroshi Ambe, Tokyo (JP); Jun Kuritani, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: Taiho Pharmaceutical Co., Ltd, Tokyo (JP)

(21) Appl. No.: 11/587,643

(22) PCT Filed: Apr. 27, 2005
(86) PCT No.: PCT/JP2005/008450

§ 371 Date: Oct. 26, 2006

Related U.S. Application Data
(60) Provisional application No. 60/566,180, filed on Apr. 29, 2004.

(51) Int. Cl. A61K 33/24; A61K 31/513; A61P 35/00; A61P 29/00; A61P 1/00

(52) U.S. Cl. 424/649; 514/274

(57) ABSTRACT

This invention provides a method for potentiating the antitumor effect of tegafur and reducing the gastrointestinal toxicity. The method comprises administering a composition containing tegafur, a DPD inhibitor and oxonic acid or a pharmaceutically acceptable salt thereof, to a patient under fasting conditions.

### TECHNICAL FIELD

The present invention relates to a method for administering Tegafur [5-Fluoro-1-[(RS)-tetrahydrofuran-2-yl]pyrimidine-2,4-(1H,3H)-dione] to reduce gastrointestinal toxicity.

### BACKGROUND ART

U.S. Pat. No. 5,155,113 teaches that a kind of pyridine compound can be used as a potentiator of the antitumor effect of 5-fluorouracil and derivatives of 5-FU. The pyridine compound sustains the concentration of 5-FU in the living body. Unfortunately, the prolonged presence of 5-FU in a living body is likely to cause inflammation in the oral cavity and gastrointestinal tissue and induce diarrhea as often experienced with continuous intravenous infusion of 5-FU alone.

U.S. Pat. No. 5,116,600 reports that oxonic acid used in combination with 5-FU or a 5-FU derivative can inhibit the occurrence of inflammation caused by 5-FU or a 5-FU derivative. However, oxonic acid reduces the antitumor effect of 5-FU or 5-FU derivatives. Therefore, oxonic acid does not produce satisfactory results in terms of potentiation of antitumor effect and alleviation of adverse effects.

U.S. Pat. No. 5,525,603, which is hereby incorporated by reference, describes compositions, methods and kits for potentiating the antitumor effect of 5-fluorouracil (hereinafter referred to as “5-FU”) by using oxonic acid or a salt thereof and a compound such as 2,4-dihydroxy-5-chloropyridine or 2,4-dihydroxy-5-cyanopyridine. Though this combination reduces the inflammation caused by administration of 5-FU, severe diarrhea, vomiting and dehydration can still occur.

As shown by the above discussed patents, 5-FU and its derivatives have often been used to treat gastrointestinal (GI) cancers, breast cancer, head and neck cancer, etc. However, when administered as a single agent, the response rate to 5-FU therapy is unsatisfactory. A number of studies, including trials modifying dosing regimens, have been conducted to improve upon the efficacy and tolerability of 5-FU therapy. Lokich et al., J. Clin. Oncl., 7, 425-432 (1989) reported their attempt to improve the clinical efficacy of 5-FU (i.e., increase its antitumor activity while reducing its adverse effects). They reported that the response rate to 5-FU was higher and the incidence of myelosuppression was lower when the drug was administered by continuous venous infusion (CVI) than when it was administered by conventional bolus infusion. The usefulness of the CVI dosing regimen for 5-FU therapy has also been found in other Phase II clinical studies [Quebbeman, E. et al., J. Surg. Oncol., 30, 60-65 (1985); Hansen, R. et al., J. Surg. Oncol., 40, 177-181 (1989); Caballero, G. A. et al., Cancer Treat. Rep., 69, 13-15 (1985); Barbounis, V. P. et al., Anticancer Res., 9, 33-39 (1989); Moynihan, T. et al., Am. J. Clin. Oncol., I 1, 461-464 (1988); Huan, S. et al., Cancer, 63, 419-422 (1989); Hansen, R. et al., Am. J. Med. Sci., 295, 91-93 (1988); Hansen, R. et al., Urology, 37, 358-361 (1991)]. In Japan, Yamao et al. reported the usefulness of 5-FU-CVI therapy [Yamao, T. et al., Jpn. J. Clin. Oncol., 25, 46-50 (1995)].

These results suggest that maintaining high serum 5-FU levels for prolonged periods of time after administration is a major factor determining the clinical efficacy of 5-FU therapy. However, dosing regimens of 5-FU that allow continued high blood 5-FU levels (e.g., CVI) are associated with several problems. One problem is the circadian variations in blood 5-FU levels [Petit, E. et al., Cancer Res., 48, 1676-1679 (1988); Harris, B. E. et al., Cancer Res., 50, 197-201 (1990); Metzger, G. et al., Clin. Pharmacol. Ther., 56, 190-201 (1994)]. It has been reported that this problem is related to dihydropyrimidine dehydrogenase (hereinafter referred to as “DPD”) and that this enzyme is an important determinant of the sensitivity of tumors to 5-FU [Fleming, R. A. et al., Cancer Res., 52, 2899-2902 (1992); Fleming, R. A. et al., Eur. J. Cancer, 29A, 740-744 (1993); Etienne, M. C. et al., J. Clin. Oncol., 12, 2248-2253 (1994); Beck, A. et al., Eur. J. Cancer, 30A, 1517-1522 (1994); Peters, G. J. et al., Eur. J. Cancer, 30A, 1408-1411 (1994); Lu, Z., et al., Cancer Res., 53, 5433-5438 (1993); Etienne, M. C. et al., J. Clin. Oncol., 13, 1663-1670 (1995)]. The second problem is the occurrence of gastrointestinal toxicity (diarrhea, nausea, vomiting, stomatitis, etc.) induced by continued high blood 5-FU levels.

Attempts have been made to prevent or reduce the adverse effects of 5-FU. In 1979, Schwartz et al., Cancer Res., 39, 3095-3101 (1979), reported that allopurinol (drug used to treat hyperuricemia) alters 5-FU and in so doing suppresses its toxic actions. Since then, clinical studies have evaluated the possibility of augmenting the antitumor activity of 5-FU and reducing its toxicity by combining 5-FU with modulators such as allopurinol [Fox, R. M. et al., Cancer Treat. Rev., 6, 143-147 (1979); Kroener, J. F. et al., Cancer Treat. Rep., 66, 1133-1137 (1982); Howell, S. B. et al., Cancer, 48, 1281-1289 (1981); Woolley, P. V. et al., J. Clin. Oncol., 3, 103-109 (1985); Ahmann, F. R. et al., Oncology, 43, 83-85 (1986)]. However, combining 5-FU with allopurinol in these clinical studies did not result in suppression of 5-FU-related toxicity or an increase in the response rate (i.e. antitumor activity). The effects of an allopurinol gargle in preventing 5-FU-induced stomatitis were also studied clinically, but sufficient evidence of such an effect was never obtained [Clark, P. I. et al., Eur. J. Surg. Oncol., 11, 267-268 (1985); Vliet, W. et al., Clin. Pharm., 8, 655-658 (1989); Loprinzi, C. L. et al., Cancer, 65, 1879-1882 (1990)].

There are two key factors which are considered to be important in addressing the goal of making 5-FU more effective and tolerable: 1) inhibition of DPD activity so that high in vivo 5-FU levels can be maintained for long periods of time, and 2) control of 5-FU metabolism in normal tissue in order to minimize adverse events.

To achieve these goals, it was considered essential to select effectors that are retained in the blood and to use at least two effective modulators at a time. If the effector and the two modulators were administered separately, it would be difficult to achieve these goals, and the toxicity of 5-FU might be increased rather than reduced. Therefore the effector and the two modulators should be administered at the same time, so that the safety of the combination of the three drugs is ensured. Tegafur (FT), a pro-drug of 5-FU, is retained in the blood for long periods when administered orally and in vivo is gradually converted into 5-FU in the presence of liver P-450. In view of this, higher blood levels of 5-FU, close to the levels achieved by CVI, were expected to be obtained by oral administration of tegafur than by oral or intravenous administration of 5-FU itself. In practice, however, the blood 5-FU levels after an oral dose of tegafur were lower than expected, due to biodegradation by DPD. Combining tegafur with a powerful DPD inhibitor achieves high blood 5-FU levels for long periods at relatively low tegafur dose levels, resulting in increased antitumor activity.

### DISCLOSURE OF THE INVENTION

The present invention is directed to a method for potentiating the antitumor effect of tegafur while reducing gastrointestinal toxicity in a patient in need of such treatment, comprising simultaneously administering to said patient a therapeutically effective amount of tegafur, a dihydropyrimidine dehydrogenase inhibitor in an amount effective for potentiating the antitumor effect and oxonic acid or a pharmaceutically acceptable salt thereof in an amount effective for suppressing gastrointestinal toxicity, under fasting conditions. Preferably, the administrations of the composition are twice over the course of a day, under fasting conditions and the dihydropyrimidine dehydrogenase inhibitor is 2,4-dihydroxy-5-chloropyridine. The two administrations of the composition are preferably separated by 6-12 hours and the fasting conditions occur at least one hour before or after a meal. More preferably the fasting conditions occur at least one hour before a meal. The 2,4-dihydroxy-5-chloropyridine and the oxonic acid or a pharmaceutically acceptable salt thereof can be used in a molar ratio of 0.4:1, preferably, tegafur, 2,4-dihydroxy-5-chloropyridine, and oxonic acid or a pharmaceutically acceptable salt thereof, are used in a molar ratio of (tegafur):(2,4-dihydroxy-5-chloropyridine):(oxonic acid or a pharmaceutically acceptable salt thereof =1:0.4:1 and the pharmaceutically acceptable salt is potassium oxonate.

In the method according to the present invention, tegafur is preferably administered at a dose of between 20 mg/m² to 45 mg/m² twice daily. Additional chemotherapy agents can be administered along with the tegafur. Such chemotherapy agents include but are not limited to cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel. Such chemotherapy agents, when used, are administered depending on the progress stage of cancer, prior treatment for the cancer, anti-cancer activity of a drug against a specific cancer type, gene expression of patients, patient's age, sex, severity of the symptoms and side effects, etc. For example, the chemotherapy agent cisplatin is preferably administered intravenously at a dose between 50-80 mg/m² daily.

The present invention can also be used for treating cancer in a mammal wherein the cancer is susceptible to 5-fluorouracil therapy, the method comprising simultaneously administering to the mammal a therapeutically effective amount of tegafur, 2,4-dihydroxy-5-chloropyridine in an amount effective for potentiating the antitumor effect of tegafur, and oxonic acid or a pharmaceutically acceptable salt thereof in an amount effective for suppressing gastrointestinal toxicity, under fasting conditions. Preferably, the administrations of the composition are twice over the course of a day, under fasting conditions and more preferably are separated by 6-12 hours, where the fasting conditions occur at least one hour before or after a meal, most preferably 1 hour before a meal. The molar ratio of (tegafur):(2,4-dihydroxy-5-chloropyridine):(oxonic acid) is preferably 1:0.4:1 and the tegafur is preferably administered in a dose of between 20 mg/m² to 45 mg/m² twice daily.

The present invention is also directed to a method for inhibiting inflammation and gastrointestinal toxicity caused by the administration of tegafur, comprising administering oxonic acid or a pharmaceutically acceptable salt thereof in an amount effective for suppressing gastrointestinal toxicity, simultaneously with said tegafur to a patient in need of such treatment, wherein said tegafur and oxonic acid or a pharmaceutically acceptable salt thereof are administered under fasting conditions. Preferably the administrations of the composition are twice over the course of a day and a dihydropyrimidine dehydrogenase inhibitor is simultaneously administered in an amount effective for potentiating the antitumor effect of tegafur.

The present invention is also directed to a method for decreasing the breakdown of oxonic acid in the gastrointestinal tract of a patient undergoing treatment with tegafur, comprising administering oxonic acid simultaneously with said tegafur to said patient under fasting conditions, preferably twice daily. The fasting conditions can be at least one hour before or after a meal. A dihydropyrimidine dehydrogenase inhibitor can be simultaneously administered in an amount effective for potentiating the antitumor effect of tegafur. The dihydropyrimidine dehydrogenase inhibitor is preferably 2,4-dihydroxy-5-chloropyridine.

The present invention is also directed to a kit for treating cancer in a mammal, comprising tegafur, 2,4-dihydroxy-5-chloropyridine, oxonic acid or a pharmaceutically acceptable salt thereof in dosages suitable for administration preferably twice a day and an additional anti-tumor agent. The additional anti-tumor agents is selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel.

The present invention is also directed to a kit for treating cancer in a mammal, comprising tegafur, 2,4-dihydroxy-5-chloropyridine, oxonic acid or a pharmaceutically acceptable salt thereof in dosages suitable for administration preferably twice a day, and optionally an additional anti-tumor agent selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel, the kit further comprising an indicator in such a form as an image printed thereon, a label attached thereto, a package insert enclosed therein or the like that reminds the patient that said tegafur, said 2,4-dihydroxy-5-chloropyridine, and said oxonic acid or a pharmaceutically acceptable salt thereof should be administered under fasting conditions.

Similarly, the present invention also provides a pharmaceutical composition for potentiating the antitumor effect of tegafur while reducing gastrointestinal toxicity in a patient in need of such treatment, comprising (A) a therapeutically effective amount of tegafur, (B) a dihydropyrimidine dehydrogenase inhibitor in an amount effective for potentiating the antitumor effect and (C) oxonic acid or a pharmaceutically acceptable salt thereof in an amount effective for suppressing gastrointestinal toxicity, and optionally an additional anti-tumor agent selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel, the pharmaceutical composition being contained in a package that comprises an indicator in such a form as an image printed thereon, a label attached thereto or a package insert enclosed therein or the like that reminds the patient that the pharmaceutical composition should be administered under fasting conditions. The administration timing and other details are those described above and those that will be described below.

### DESCRIPTION OF THE INVENTION

The present invention provides a method for potentiating the antitumor effect of an antitumor composition containing a therapeutically effective amount of tegafur while suppressing the gastrointestinal side effects of the antitumor composition by administering the antitumor composition under fasting conditions in order to avoid any undesirable effects of food on the bioavailability of the active components.

In a preferred embodiment of the present invention, S-1, an oral pyrimidine fluoride-derived anti-cancer agent containing Tegafur [5-Fluoro-1-[(RS)-tetrahydrofuran-2-yl]pyrimidine-2,4-(1H,3H)-dione] (FT) in combination with 2 classes of modulators: 5-chloro-2,4-dihydroxypyridine (CDHP) and potassium oxonate (Oxo), is administered, typically twice a day, under fasting conditions to reduce gastrointestinal toxicity while maintaining antitumor efficacy. S-1 (as described in U.S. Pat. No. 5,525,603) was developed to enhance the clinical advantage of 5-FU and ameliorate its disadvantages (i.e., gastrointestinal toxicity), and was tested by being administered after meal in the US and Europe in the same way as in Japan where S-1 was already approved, but unfortunately still produces severe diarrhea which can result in dehydration if administered under non-fasting conditions twice a day.

The purpose of combining three drugs (FT, CDHP, and Oxo) in the formulation of S-1 was to achieve high clinical efficacy by reducing the gastrointestinal toxicity and improving tolerability. The contributions of each of the three components of S-1 are outlined below:
FT is a prodrug of 5-FU, with excellent oral bioavailability. It is gradually converted to 5-FU in vivo.
CDHP inhibits the catabolism of 5-FU by reversibly inhibiting the enzyme of dihydropyrimidine dehydrogenase (DPD). CDHP is 180-fold more potent than uracil. CDHP helps maintain effective blood and tumor concentrations of 5-FU for a prolonged period thus potentially achieving a similar therapeutic effect as protracted infusion of 5-FU.
Oxo blocks phosphorylation of 5-FU by orotate phosphoribosyltransferase (OPRT) in the gastrointestinal tract. Oxo shows a toxicity-reducing action on the 5-FU provoked gastrointestinal disturbances through its distribution in gastrointestinal tissues at high concentrations after oral administration. Oxo is acid labile and thus its bioavailability is affected by food intake.

Tegafur (FT) is converted to 5-FU which in itself is not cytotoxic. 5-FU has to be metabolized in the cell to exert its antitumor effects via FdUMP (2′-deoxy-5-fluorouridine-5′-monophosphate) to block TS (thymilate synthase). 90% of the administered 5-FU is rapidly catabolized mainly in the liver into inactive metabolites by the enzyme DPD. Preclinical data indicate that the 5-FU cytotoxicity can be increased significantly by blocking DPD using CDHP. Clinical data also indicate that S-1 plasma-5-FU levels are comparable to plasma-5-FU levels obtained by continuous infusion of 5-FU [Yamada Y. et al., Br. J. Cancer, 2003 Sep. 1; 89(5):816-20.] due to the activity of CDHP. DPD activity may be a key in the proper application of chemotherapeutics in cancer treatment. Based on a population study [Fukushima M, et al., Int. J. Mol. Med, 2003; 12:839-844.] reviewing data from 2,592 clinically removed tumors (1112: colon, 724: gastric, 520: breast and 236: NSCLC), 61% of patients with gastric cancer, and 40% of patients with colorectal cancer were categorized as having tumors that expressed relatively high DPD activity. It is hypothesized that conventional 5-FU or its derivatives would be less effective in patients with high DPD activity than in patients with low DPD activity because 5-FU would be quickly metabolized by the high DPD activity. S-1 is expected to be effective for the treatment of gastric cancer patients not only in patients with low DPD activity, but also with high DPD activity as well due to the properties of CDHP (one component of S-1) of being a DPD inhibitor.

Without a DPD inhibitor, 90% of 5-FU administered is rapidly catabolized by DPD primarily in the liver and excreted in the urine as F-β-Ala (FBAL). FBAL has been implicated as the cause of neurotoxicity and cardiotoxicity associated with 5-FU administration. An important finding of DPD inhibitor studies is the low incidence of hand foot syndrome (HFS) [Milano, et al., Eur. J. Cancer., 36, 37-42 (2000)].

Accordingly the present invention provides a method for treating a cancer susceptible to 5-fluorouracil therapy in a mammal, comprising simultaneously administering to the mammal a composition comprising a therapeutically effective amount of tegafur, an antitumor effect-potentiating effective amount of a DPD inhibitor, and a side effect-suppressing effective amount of oxonic acid or a pharmaceutically acceptable salt thereof, wherein said composition is administered under fasting conditions, typically twice a day. The language “fasting conditions” as used in the present document is intended to mean at least one hour before or after a meal.

In other words, the present invention particularly provides, in a method of treating a cancer susceptible to tegafur therapy in a patient in need of such treatment a method comprising administering to the patient a therapeutically effective amount of tegafur, a DPD inhibitor, and oxonic acid or a pharmaceutically acceptable salt thereof, the improvement comprising administering the composition under fasting conditions, typically twice a day.

The tegafur for use in the present invention is a known compound and can be prepared by conventional processes as disclosed, e.g. in U.S. Pat. No. 5,525,603 and Japanese Examined Patent Publication No. 10510/1974.

DPD inhibitors are known and can be easily prepared by conventional processes. Preferred DPD inhibitors include 2,4-dihydroxy-5-chloropyridine, and 2,4-dihydroxy-5-cyanopyridine.

Oxonic acid per se is a known compound. Useful pharmaceutically acceptable salts thereof include acid addition salts and basic compound salts. Examples of useful acids capable of forming the acid addition salts are hydrochloric acid, sulfuric acid, phosphoric acid, hydrobromic acid and like inorganic acids, oxalic acid, succinic acid, maleic acid, fumaric acid, malic acid, tartaric acid, citric acid, malonic acid, methanesulfonic acid, benzoic acid and like organic acids. Examples of useful basic compounds capable of forming the pharmaceutically acceptable basic compound salts are sodium hydroxide, potassium hydroxide, calcium hydroxide, sodium carbonate, potassium hydrogencarbonate, etc. Substances capable of producing oxonic acid in the living body, such as ester derivatives of oxonic acid, are usable as the oxonic acid.

Co-administration of food with S-1 was found to affect the pharmacokinetics of some of the S-1 constituents (e.g. the rate, but not the extent of absorption of tegufur and CDHP were influenced). Since toxicity of S-1 is related to the area under the curve (AUC, a measure of the total amount of drug that remains in circulation) of 5-FU [Van Groeningen C. J., Peters G. J., Schornagel J. H., Gall H., Noordhuis P., De Vries M., Turner S. L., Swart M. S., Pinedo H. M., Hanauske A. R. and Giaccone G. Phase I clinical and pharmacokinetic study of oral S-1 in patients with advanced solid tumors. J. Clin. Oncol., 18: 2772-2779, 2000], and since the bioavailability of 5-FU is within the FDA confidence intervals, it is unlikely that the safety of S-1 will be affected by food. Food did not affect the AUC of CDHP. Similarly the accumulation of uracil, an indirect result of DPD inhibition was only marginally affected. However, oxonic acid added to inhibit phosphoribosylation of 5-FU to its active metabolite FUMP (5-fluorouridine-5′-monophosphate) specifically in gut mucosa, showed a greater degradation in the co-administration of food with S-1. This indicates that it might accumulate to a lesser extent in the gut mucosa, decreasing its protective effect.

Previous studies with oral fluoropyrimidine formulations reported an increase in the time-period for absorption when food was given. Non-compartmental methods focusing on Cmax, Tmax and AUC-_{INF} were used in these [Shepard D. R., Mani S., Kastrissios H., Learned-Coughlin S., Smith D., Magnum P. E. S. M., Janisch L., Fleming G. F., Schilsky R. L. and Ratain M. J. Estimation of the effect of food on the disposition of oral 5-fluorouracil in combination with eniluracil. Cancer Chemother. Pharmacol., 49: 398-402, 2002; Reigner B., Verweij J., Dirix L., Cassidy J., Twelves C., Allman D., Weidekamm E., Roos B., Banken L., Utoh M. and Osterwalder B. Effect of food on the pharmacokinetics of capecitabine and its metabolites following oral administration in cancer patients. Clin. Cancer Res., 4: 941-948, 1998] and a study [Godefridus J. Peters, Paul Noordhuis, Cornelis J. van Groeningen, Giuseppe Giaccone, Ulbe Holwerda, Daphne Voorn, Ad Schrijvers, Jan H. Schornagel, Jos H. Beijnen, Pierre Fumoleau, and Jan H. M. Schellens The Effect of food on the pharmacokinetics of S-1 after single oral administration on patients with solid tumors. Clinical Cancer Research 10: 4072-4076, 2004, published on Jun. 15, 2004]. For capecitabine [Reigner B., Verweij J., Dirix L., Cassidy J., Twelves C., Allman D., Weidekamm E., Roos B., Banken L., Utoh M. and Osterwalder B. Effect of food on the pharmacokinetics of capecitabine and its metabolites following oral administration in cancer patients. Clin. Cancer Res., 4: 941-948, 1998] the prodrug itself and its first metabolite, showed a clear difference between the fed and fasting patients, but 5-FU was only affected marginally. In an Eniluracil (EU)-5-FU formulation, food decreased 5-FU absorption possibly because 5-FU itself was administered [Shepard D. R., Mani S., Kastrissios H., Learned-Coughlin S., Smith D., Magnum P. E. S. M., Janisch L., Fleming G. F., Schilsky R. L. and Ratain M. J. Estimation of the effect of food on the disposition of oral 5-fluorouracil in combination with eniluracil. Cancer Chemother. Pharmacol., 49: 398-402, 2002]. Irreversible suicide inactivation of DPD prevents any degradation of 5-FU; the major elimination pathway of 5-FU. Without EU the Km for elimination of 5-FU equals the Km of 5-FU for DPD [Collins J. M., Dedrick R. L., King F. G., Speyer J. L. and Myers C. E. Non-linear pharmacokinetic models for 5-fluorouracil in man. Clin. Pharmacol. Therap., 28: 235-246, 1980], but with EU this Km could not be calculated, and elimination no longer followed Michaelis-Menten kinetics anymore, leaving a linear component. With S-1, which contains a reversible DPD inhibitor, elimination of 5-FU is still biphasic, meaning that degradation still contributes to 5-FU's elimination, independent of food status.

The bioavailability of the other S-1 constituents has not previously been described. Since CDHP is not metabolised, its elimination will be by urinary excretion [Peters G. J., Noordhuis P., Van Kuilenburg A. B. P., Schornagel J. H., Gall H., Turner S. L., Swart M. S., Voorn D., Van Gennip A. H., Wanders J., Holwerda U., Smid K., Giaccone G., Fumoleau P. and Van Groeningen C. J. Pharmacokinetics of S-1, an oral formulation of tegafur, oxonic acid and 5-chloro-2,4-dihydroxypyridine (molar ratio 1:0.4:1) in patients with solid tumors. Cancer Chemother. Pharmacol., 38: 1-12, 2003; Hirata K., Horikoshi N., Okazaki M., Denno R., Sasaki K., Nakano Y., Ishizuka H., Yamada Y., Uno S., Taguchi T., and Shirasaka T. Pharmacokinetic study of S-1, a novel oral fluorouracil anti-tumor drug. Clin. Cancer Res. 5: 2000-2005, 1999]. In experimental renal failure models plasma clearance of CDHP was retarded [Ikeda M., Furukawa H., Imamura H., Shimizu J., Ishida H., Masutani S., Tatsuta M., Kawasaki T. and Satomi T. Pharmacokinetic study of S-1, a novel oral fluorouracil antitumor agent in animal model and in patients with impaired renal function. Cancer Chemother. Pharmacol. 50: 25-32, 2002] in line with the degree of renal impairment. Also in patients with renal dysfunction CDHP clearance was prolonged, leading to delayed t½ and higher AUC of 5-FU [Ikeda M., Furukawa H., Imamura H., Shimizu J., Ishida H., Masutani S., Tatsuta M., Kawasaki T. and Satomi T. Pharmacokinetic study of S-1, a novel oral fluorouracil antitumor agent in animal model and in patients with impaired renal function. Cancer Chemother. Pharmacol. 50: 25-32, 2002], possibly due to a retained DPD inhibition following a prolonged exposure.

In European and US studies diarrhea was the dose-limiting toxicity in contrast to in Japan, where the formulation is registered and in clinical use with an efficacy similar to other oral formulations. Current clinical studies in the US and Europe focus on decreasing diarrhea by using another schedule such as a two-week schedule since diarrhea usually occurs in the last period of administration. Gastro-intestinal side effects of S-1 are less common in Japanese patients [Hirata K., Horikoshi N., Okazaki M., Denno R., Sasaki K., Nakano Y., Ishizuka H., Yamada Y., Uno S., Taguchi T., and Shirasaka T. Pharmacokinetic study of S-1, a novel oral fluorouracil anti-tumor drug. Clin. Cancer Res. 5: 2000-2005, 1999; Ohtsu A., Baba H. and Sakata Y. Phase II study of S-1, a novel oral fluoropyrimidine derivative, in patients with metastatic colorectal carcinoma. S-1 cooperative Colorectal Carcinoma Study Group. Br. J. Cancer, 83: 141-145, 2000] compared to Europeans [Van den Brande J., Schoffski P., Schellens J. H., Roth A D, Duffaud F., Weigang-Kohler K., Reinke F., Wanders J., De Boer R. F., Vermorken J. B. and Fumoleau P. EORTC Early Clinical Studies Group early phase II trial of S-1 in patients with advanced or metastatic colorectal cancer. Br. J. Cancer, 88: 648-53, 2003; Van Groeningen C. J., Peters G. J., Schornagel J. H., Gall H., Noordhuis P., De Vries M., Turner S. L., Swart M. S., Pinedo H. M., Hanauske A. R. and Giaccone G. Phase I clinical and pharmacokinetic study of oral S-1 in patients with advanced solid tumors. J. Clin. Oncol., 18: 2772-2779, 2000]. This might be due to potential ethnic and cultural differences (e.g. types of foods consumed, different expression of degradative enzymes, different GI absorption, etc.). Therefore, the present invention intends to decrease the gastro-intestinal side effects of S-1, and is especially useful for patients who suffer from gastro-intestinal side effects of S-1 such as diarrhea, and is typically beneficial for Caucasian patients.

Food had a profound effect on the bioavailability of oxonic acid, leading to a rapid increase in the concentration of cyanuric acid. This might decrease its anticipated protection against the gastro-intestinal side effects of 5-FU, since oxonic acid accumulates specifically in normal gut cells [Shirasaka T., Shimamoto Y. and Fukushima M. Inhibition by oxonic acid of gastrointestinal toxicity of 5-fluorouracil without loss of its antitumor activity in rats. Cancer Res., 53: 4004-4009, 1993] to prevent 5-FU metabolism to toxic metabolites. If administered along with a relatively heavy meal, protection might be limited.

The antitumor effect-potentiating method of the present invention can be carried out by formulating tegafur, the DPD inhibitor and oxonic acid or a pharmaceutically acceptable salt thereof into a single preparation (i.e. antitumor composition) and administering the composition, or by administering the separate compounds simultaneously. The combined preparation or the separate compounds are administered, typically twice daily, under fasting conditions, at least one hour and preferably 1-2 hours before or after a meal.

The pharmaceutical composition may be contained in a package that comprises an indicator in such a form as an image printed thereon, a label attached thereto, a package insert enclosed therein or the like that shows the timing of administration (namely, the pharmaceutical composition should be administered under fasting conditions) and optionally other information relating to the regimen described above.

Additional anti-tumor agents selected from the group consisting of cisplatin, docetaxel, paclitaxel, 1,3-bis(2-chloroethyl)-1-nitrosourea, doxorubicin, epirubicin, etoposide, methotrexate, mitomycin, gemcitabine, carboplatin, gefitinib, pemetrexed, Avastin, and Cetuximab, can be administered along with the tegafur in the present invention. S-1 has been studied in combination with cisplatin for advanced gastric cancer in Japan. The objective of the study was to identify the MTD (Maximum Tolerated Dose) of cisplatin when administered in combination with a fixed dose of S-1. The S-1 dose actually taken was similar to the late Phase II studies (mean 36.0 mg/m², range 31.8-39.6 mg/m²) and administered twice daily for 21 days with a 14-day recovery period. Cisplatin was infused on Day 8 of a cycle at doses of 60 mg/m², or 70 mg/m². The cycle was repeated every 35 days. At the 70 mg/m² dose level, 1 patient developed Grade 4 neutropenia and another patient Grade 4 anorexia (of 6 patients). The recommended dose for the phase II portion was determined to be the first dose level of 60 mg/m². Objective responses were seen in 19 of 25 patients, evaluated yielding a 76% response rate. The PK (Pharmacokinetics) portion of the study indicates that there is no interaction between cisplatin and S-1.

Though such anti-tumor agents would be administered to the patient receiving tegafur, a DPD inhibitor, and oxonic acid or a pharmaceutically acceptable salt thereof, these agents would likely be administered at different times than the tegafur (i.e. not always simultaneously).

It is preferable to use about 0.1 to about 10 moles, preferably about 0.5 to about 5 moles, of oxonic acid or a pharmaceutically acceptable salt thereof, per mole of the DPD inhibitor for potentiating the antitumor effect of tegafur and reducing gastrointestinal toxicity. Particularly good results can be obtained by administering about 0.1 to about 5 moles, preferably about 0.1 to about 1.5 moles, of the DPD inhibitor and about 0.1 to about 5 moles, preferably about 0.2 to about 2 moles, of oxonic acid or a pharmaceutically acceptable salt thereof, per mole of tegafur. The three active ingredients can be admixed with a pharmaceutically acceptable carrier to provide a preparation in an optional unit dosage form which is then administered.

The foregoing pharmaceutical compositions are prepared in non-injection form by the conventional method using a suitable pharmaceutically acceptable carrier. Examples of useful carriers are those widely used in the manufacture of conventional pharmaceutical compositions, such as fillers, extenders, binders, disintegrators, surfactants, lubricants and like diluents and excipients. The preferred administration route is oral.

There is no particular restriction on the unit dosage form which can be adopted for the antitumor effect-potentiating composition (i.e., a composition for potentiating anti-cancer composition of tegafur, the composition comprising a DPD inhibitor such as CDHP) or antitumor composition of the invention in the treatment of malignant tumors in mammals inclusive of human beings insofar as it is in non-injection form. Thus, optional desired unit dosage form can be selected according to the purpose of treatment. Examples thereof are oral dosage forms such as tablets, coated tablets, pills, powders, granules, capsules, solutions, suspensions, emulsions, etc. and parenteral dosage forms such as suppositories, ointments, plasters, etc. These dosage forms can be manufactured by conventional pharmaceutical procedures known in this field.

As the carrier for shaping into the form of tablets, there can be employed various excipients such as lactose, sucrose, sodium chloride, glucose, urea, starch, calcium carbonate, kaolin, crystalline cellulose, silicic acid, etc.; binders such as simple syrup, glucose solution, starch solution, gelatin solution, carboxymethylcellulose, shellac, methylcellulose, potassium phosphate, polyvinylpyrrolidone, gum arabic powder, gum tragacanth powder, etc.; disintegrators such as dry starch, sodium alginate, agar powder, laminaran powder, sodium hydrogen carbonate, calcium carbonate, polyoxyethylene-sorbitan fatty acid esters, sodium lauryl sulfate, stearic acid monoglyceride, starch, lactose, etc.; antidisintegrators such as sucrose, stearic acid, cacao butter, hydrogenated oil, etc.; absorption promotors such as quaternary ammonium bases, sodium lauryl sulfate, etc.; humectants such as glycerol, starch, etc.; adsorbents such as starch, lactose, kaolin, bentonite, colloidal silicic acid, etc.; and lubricants such as purified talc, stearic acid salts, boric acid powder, polyethylene glycol, etc. Where necessary, the tablets may be in the form of coated tablets such as sugar-coated tablets, gelatin-coated tablets, enteric-coated tablets, film-coated tablets, or double or multi-layer tablets, etc.

Carriers for shaping into the form of suppositories include but are not limited to polyethylene glycol, cacao butter, higher alcohols, esters of higher alcohols, gelatin, semi-synthetic glycerides, etc.

Capsules are manufactured by mixing the antitumor composition with any of the carriers mentioned above and encapsulating the mixture in hard gelatin capsule, soft capsule or other capsules.

For manufacturing in the form of pastes, creams, and gels, there is employed a diluent such as, for example, white petrolatum, paraffin, glycerol, cellulose derivatives, polyethylene glycols, silicone, bentonite, etc.

When required, the above preparations may contain coloring agents, preservatives, perfumes, flavors, sweeteners, other medicament, etc.

The amounts of tegafur, the DPD inhibitor and oxonic acid or a pharmaceutically acceptable salt thereof, which are the active ingredients used in the invention, are dependent on the dosage form, route of administration, dosing schedule, etc. and can be appropriately chosen without specific limitation. Generally, however, the total amount of active ingredients in the dosage form may range from about 1 to about 70 percent by weight.

In the present invention, the dosage of each active ingredient can be selected according to the method of administration, the patient's age, sex and other factors, the degree of disease and so on. In the case of oral administration, the standard dosage for a human adult is usually about 0.1 to about 100 mg/kg/day, preferably about 1 to about 30 mg/kg/day, for tegafur, about 0.1 to about 100 mg/kg/day, preferably about 1 to about 50 mg/kg/day, for the DPD inhibitor and about 0.1 to about 100 mg/kg/day, preferably about 1 to about 40 mg/kg/day, for oxonic acid or a pharmaceutically acceptable salt thereof. The composition of the invention is typically administered twice daily at an interval of 6 to 12 hours under fasting conditions. In the case of suppositories, for human adults, the equivalent of about 1 to 100 mg/kg/day of tegafur is administered into the rectum twice a day at an interval of 6 to 12 hours.

In the case of oral administration of S-1 (namely, a composition comprising tegafur, CDHP and Oxo in a tegafur:CDHP:Oxo molar ratio of 1:0.4:1), the tegafur is preferably administered in a dose of between 20 mg/m² to 45 mg/m² twice daily.

The dose for a patient is usually determined based on the body surface area (BSA) calculated from the height and weight of the patient. The calculation of the body surface area is carried out using a conventional suitable method depending on the race, sex, health condition, symptom, and the like of the patient, for example, using one of the following calculating formulas under items (1) to (6) below, preferably using the calculating formula under item (2)(b) or item (6). For example, the calculating formula under item (6) below can be used for calculating BSA of Asian patients especially Japanese patients and the calculating formula under item (2) (b) below can be used for calculating BSA of Caucasian patients.

(1) The Mosteller formula (See N. Engl. J. Med. 1987 Oct. 22; 317 (17): 1098 (letter))

BSA(m²)=([Height (cm)×Weight (kg)]/3600)^{1/2}

(2) The DuBois and DuBois formula (See Arch. Int. Med. 1916 17: 863-71; J. Clin. Anesth. 1992; 4 (1): 4-10)

BSA(m²)=0.20247×Height (m)^{0.725}×Weight (kg)^{0.425} (a)

BSA(m²)=0.007184×Height (cm)^{0.725}×Weight (kg)^{0.425} (b)

(3) The Haycock formula (See The Journal of Pediatrics 1978 93:1: 62-66)

BSA(m²)=0.024265×Height (cm)^{0.3964}×Weight (kg)^{0.5378}

(4) The Gehan and George formula (See Cancer Chemother. Rep. 1970 54: 225-35)

BSA(m²)=0.0235×Height (cm)^{0.42246}×Weight (kg)^{0.51456}

(5) The Boyd formula (See Minneapolis: University of Minnesota Press, 1935)

BSA(m²)=0.0003207×Height (cm)^{0.3}×Weight (gram)^{(0.7285-(0.0188×LOG (gram))}

(6) The Fujimoto formula (See Nippon Eiseigaku Zasshi 1968; 5:443-50)

BSA(cm²)=88.83×Height(cm)^{0.663}×Weight(kg)^{0.444}

For example, when the body surface area of a cancer patient 175 cm high and 70 kg in weight is calculated by the above formula of item (2)(b), the area is determined to be [0.007184×175(cm)×70(kg)=1.85(m²).

In the case of oral administration of S-1, the standard dosage for a human adult is usually about 40 to about 300 mg/day, preferably about 50 to about 200 mg/day, for tegafur, about 10 to about 90 mg/day, preferably about 14 to about 60 mg/day, for CDHP and about 38 to about 293 mg/day, preferably about 48 to about 195 mg/day, for Oxo.

The route of administration of the antitumor composition of the present invention is not specifically limited insofar as it is non-injection route and may be, for example, intestinal, oral, rectal, stomatic, percutaneous or the like and can be selected according to the dosage form, the patient's age, sex and other factors, the severity of the symptom of the patient and so on. However, oral administration is preferred.

The tegafur, the DPD inhibitor and the oxonic acid or a pharmaceutically acceptable salt thereof, can be provided in the form of a kit. The components can be in separate containers or some or all of the components can be combined in a suitable formulation. Whether provided as separate components or as a formulation, the components are in dosage units suitable for administration typically twice a day. The kit may further comprise an indicator in such a form as an image printed thereon, a label attached thereto, package insert enclosed therein or the like that shows the timing of administration (namely, the pharmaceutical composition should be administered under fasting conditions) and optionally other information relating to the regimen described above.

Additional anti-tumor agents described above can also be included in the kit. Though such anti-tumor agents would be administered to the patient receiving tegafur, a DPD inhibitor, and oxonic acid or a pharmaceutically acceptable salt thereof, these agents would likely be administered at different times than the tegafur (i.e. not always simultaneously).

In preclinical studies, S-1 showed a tumor proliferation-inhibitory action at lower doses than other conventional pyrimidine fluoride-derived anticancer agents, such as 5-FU. In the 5-FU provoked diarrhea model in Cynomolgus monkeys and beagle dogs, S-1 showed a marked inhibitory action on the development of diarrhea when administered at specific times. On the basis of the results of the preclinical studies, S-1 is expected to be useful in the treatment of several types of malignant solid tumors. S-1 is currently being tested for the treatment of gastric, esophagus, colorectal, breast, NSCLC, head & neck, pancreatic, biliary tract, renal cell and prostate cancer in Japan. S-1 is also expected to be useful for the treatment of cancers of the liver, gallbladder-bile duct, urinary bladder, uterine, cervix, and so on.

S-1 was approved for use in Japan for the treatment of gastric cancer in January 1999, in April of 2001 for head and neck cancer, in December of 2003 for colorectal cancer, and in December 2004 for NSCLC. S-1 was approved in South Korea for the treatment of gastric cancer and head and neck cancer in July 2003, and for colorectal cancer in September 2004 based on Japanese data.

The present invention will be described in more detail with reference to the following examples. These examples are intended to illustrate but not limit the present invention.

### EXAMPLES

### (1) S-1 Administered Orally, Twice a Day, After Meal

A Phase I study has been conducted in the US under IND #53,765. This was a 35-day study (28-day treatment period, 7-day rest period). S-1 was administered orally, twice daily, after meal, for 28 consecutive days in a total of 16 patients. The dose levels were 30 mg/m², 35 mg/m², and 40 mg/m². Three patients were to be studied at each dose level before escalating doses, and an additional 10 patients were to be treated at the maximum dose in order to fully define the effects of S-1.

The antitumor effect of S-1 was determined by measuring the reduction in tumor size. A partial response was documented in one patient with colorectal cancer. Fourteen (87.5%) patients discontinued the study, the majority (11; 68.8%) from disease progression. One (6.3%) patient discontinued due to an adverse event (AE) and 2 (12.5%) patients discontinued due to unacceptable toxicity. Two (12.5%) patients died within 30 days of the last dose of S-1 and were considered to be unlikely to be related to S-1 therapy. During the study, 8 (50.0%) patients experienced serious adverse events (SAEs). Serious adverse events considered to be related to S-1 included diarrhea, dehydration, and vomiting. The MTD of S-1 was determined to be 30 mg/m². Dose-limiting toxicities (DLTs) during Course 1 included the following: diarrhea, dehydration, abdominal pain, bilirubinemia, and the inability of the patient to take =75% of the planned chemotherapy. The toxicity was reversible in almost all cases by interrupting or discontinuing S-1 therapy. At the MTD, the majority ( 7/9; 77.8%) of patients experienced AEs of maximum toxicity Grade 2 or lower during Course 1, showing that S-1 was well tolerated at the MTD of 30 mg/m².

### (2) Determination of Dose Limiting Toxicity

Based on the results of Japanese studies with S-1, the first dose studied was 25 mg/m² twice daily, after meal in a phase I study in Europe. No dose-limiting toxicity occurred in six patients who were treated at this dose. Five patients were then treated with 45 mg/m². This dose was determined to be the MTD, with severe diarrhea as the main dose-limiting toxicity. Another six patients were treated with a dose of 35 mg/m²; no severe toxicity was observed. In six patients on 40 mg/m², the last dose level studied, severe diarrhea was again observed (mainly in patients who had received extensive prior chemotherapy). To evaluate the safety of this dose level in patients who had not received extensive prior chemotherapy, another five patients who had not been extensively pre-treated, were included.

### <Results>

Treatment with 25 mg/m² of S-1 twice daily showed mild toxicity. At 45 mg/m² twice daily, diarrhea (Grade 3 in 1 patient, Grade 4 in 2 patients) was the DLT. Other severe toxicities were Grade 4 vomiting (1 patient), Grade 3 anorexia (2 patients), Grade 3 fatigue/malaise (2 patients) and Grade 3 leukopenia/neutropenia (1 patient). No severe toxicity was observed at 35 mg/m² twice daily. At 40 mg/m², diarrhea was the DLT for patients who had received extensive prior chemotherapy: of 3 patients who had been heavily pre-treated with chemotherapy, 1 patient had Grade 3 and 1 patient had Grade 4 diarrhea. In eight patients who had received no extensive prior chemotherapy, one case of Grade 4 diarrhea and one case of Grade 3 malaise was reported. Therefore, 45 mg/m² twice daily is the MTD for patients without extensive prior chemotherapy treatment, with diarrhea as the DLT. The recommended dose for future ECSG/NDDO Phase II studies in colorectal and gastric cancer patients who have received no prior, or only 5-FU adjuvant, chemotherapy is 40 mg/m² twice daily of S-1. For patients who have been more heavily pre-treated with chemotherapy the MTD is 40 mg/m² twice daily (diarrhea as the DLT), with a recommended dose of 35 mg/m² S-1 twice daily for treatment of this category of patients.

### (3) Food Effect PK Study

The effects of food on PK parameters were examined at a dose of 35 mg/m² of S-1. 17 patients were enrolled and administered S-1 both after breakfast and before breakfast in cross-over manner. The patient's diagnosis were CRC (7), breast (3), gastric (3) and other (4). The results suggested that Oxo is more stable in fasting conditions (CA, i.e., Cyanuric acid, is an inactive metabolite of Oxo). Based on this data, S-1 is administered at least 1 hour before meals in ongoing US studies.

### <Patients and Methods>

A total of 17 patients were entered in this pharmacokinetic study after informed consent. All patients were randomized for the two arms of the study and received S-1 at 35 mg/m².

In sequence A, patients received S-1 on day-7 without breakfast and on day 0 after breakfast. In sequence B, patients received S-1 after breakfast at day-7 and on day 0 without breakfast.

Blood was sampled during 24 hours. Samples were taken before and at 30 min, 1 hr, 2 hr, 4 hr, 8 hr, 10 hr and 24 hr after S-1 intake. Blood samples were taken by venipuncture in heparinized tubes of 9 ml and transported on ice for immediate processing.

Blood samples were centrifuged for 5 min at 4000 rpm and 4° C. The plasma was transferred to 2 eppendorf vials of 2 ml and stored at −20° C.

Analysis for tegafur and cyanuric acid was performed by validated HPLC assays, while the analysis of the other compounds was performed after derivatization by validated gas-chromatography-mass spectrometry methods as described previously [Peters G. J., Noordhuis P., Van Kuilenburg A. B. P., Schornagel J. H., Gall H., Turner S. L., Swart M. S., Voorn D., Van Gennip A. H., Wanders J., Holwerda U., Smid K., Giaccone G., Fumoleau P. and Van Groeningen C. J. Pharmacokinetics of S-1, an oral formulation of tegafur, oxonic acid and 5-chloro-2,4-dihydroxypyridine (molar ratio 1:0.4:1) in patients with solid tumors. Cancer Chemother. Pharmacol., 38: 1-12, 2003.].

### <Pharmacokinetic Evaluation>

The pharmacokinetic parameters for tegafur, 5FU, oxonic acid, cyan uric acid, and CDHP were calculated with the computer program WinNonLin (version 1.5). The data were analyzed using non-compartment modeling. The parameters that could be established were: time point of maximum observed concentration in plasma (Tmax), concentration in plasma corresponding to Tmax (Cmax), terminal half-life (t½), area under the plasma concentration versus time (C-t) curve (AUCall).

### (4) Results

### <Pharmacokinetics of Tegafur>

The parent compound, tegafur, reached its Tmax earlier without breakfast (Table 1). The Cmax was higher in the fasting group than the feeding group. There was no significant difference for the t½ and AUCall in either group.

### <Pharmacokinetics of 5-FU>

The Tmax of 5-FU was reached earlier without breakfast. There was no significant difference for the t½, Gmax and AUCail in either group.

### <Pharmacokinetics of CDHP>

The Tmax of CDHP was also reached earlier without breakfast. There was no significant difference for the t½, Cmax and AUCall in either group.

### <Pharmacokinetics of Oxonic Acid (Oxo)>

The Tmax of Oxonic acid was reached significantly faster without breakfast. The corresponding Cmax was significantly higher without breakfast. The t½ was not significantly different in either group. AUCall of Oxo was higher in the fasting group than the feeding group.

### <Pharmacokinetics of Cyanuric Acid (CA)>

Cyanuric acid showed a faster Tmax without breakfast. The corresponding Cmax was not significantly different in either group. Also the t½ was not significantly different in either group. AUCall of cyanuric acid showed higher values in the feeding group as compared to the fasting group.

TABLE 1
Pharmacokinetic parameters of each analyte after oral administration of S-1 to fasting or
feeding patients.

| | | AUCall | | | | | | | |
|---|---|---|---|---|---|---|---|---|---|
| | | (hr · ng/mL) | | Cmax (ng/mL) | | Tmax (hr) | | t½ (hr) | |
| | | fasted | fed | fasted | fed | fasted | fed | fasted | fed |
| FT | Mean | 15737.8 | 14817.7 | 2374.1 | 1457.2 | 0.6 | 2.1 | 8.6 | 8.6 |
| | SD | 3696.6 | 3092.1 | 628.6 | 288.1 | 0.2 | 0.8 | 1.7 | 3.2 |
| | Count | 17 | 17 | 17 | 17 | 17 | 17 | 17 | 17 |
| 5-FU | Mean | 1099.7 | 1374.6 | 210.2 | 315.2 | 1.8 | 2.7 | 2.2 | 2.6 |
| | SD | 268.2 | 1623.0 | 53.0 | 525.4 | 0.5 | 1.1 | 1.7 | 1.9 |
| | Count | 17 | 17 | 17 | 17 | 17 | 17 | 17 | 16 |
| CDHP | Mean | 1919.4 | 1685.4 | 464.0 | 328.1 | 1.1 | 2.2 | 4.5 | 5.2 |
| | SD | 380.8 | 828.3 | 121.4 | 260.9 | 0.4 | 0.9 | 2.2 | 3.3 |
| | Count | 17 | 17 | 17 | 17 | 17 | 17 | 17 | 17 |
| Oxo | Mean | 808.7 | 349.9 | 158.1 | 62.3 | 1.6 | 2.4 | 3.9 | 4.2 |
| | SD | 341.9 | 165.5 | 73.2 | 36.2 | 0.5 | 0.9 | 2.0 | 4.7 |
| | Count | 17 | 17 | 17 | 17 | 17 | 17 | 17 | 17 |
| CA | Mean | 517.3 | 1220.9 | 117.8 | 160.2 | 1.3 | 3.5 | 5.3 | 6.7 |
| | SD | 571.6 | 818.4 | 129.2 | 119.6 | 1.0 | 1.0 | 6.3 | 7.2 |
| | Count | 17 | 17 | 17 | 17 | 14 | 15 | 12 | 14 |

CA: cyanuric acid

Patients were treated with 35 mg/m² in fasted or fed condition. The results summarized in Table 1 show better absorption of each S-1 component and increased 5-FU levels due to higher absorption of CDHP (DPD inhibitor) when administered before meals.

The results also suggest that Oxo is more stable in fasting conditions (CA is an inactive metabolite of Oxo). Therefore, under fasting conditions, it is expected that Oxo shows a toxicity-reducing action on the 5-FU provoked gastrointestinal disturbances satisfactorily.

Based on this data, S-1, when administered under fasting conditions, achieves excellent advantages.

### INDUSTRIAL APPLICABILITY

According to the invention, excellent advantages, such as decreased gastrointestinal toxicity and diarrhea, compared to the treatment schedule of after meal administration, longer term treatment due to lower incidence of diarrhea, are achieved by simultaneously administering to a patient (A) a therapeutically effective amount of tegafur, (B) a dihydropyrimidine dehydrogenase inhibitor in an amount effective for potentiating the antitumor effect and (C) oxonic acid or a pharmaceutically acceptable salt thereof in an amount effective for suppressing gastrointestinal toxicity, under fasting conditions.

1. A method for potentiating the antitumor effect of tegafur while reducing gastrointestinal toxicity in a patient in need of such treatment, comprising simultaneously administering to said patient (A) a therapeutically effective amount of tegafur, (B) a dihydropyrimidine dehydrogenase inhibitor in an amount effective for potentiating the antitumor effect and (C) oxonic acid or a pharmaceutically acceptable salt thereof in an amount effective for suppressing gastrointestinal toxicity, under fasting conditions.

2. The method according to claim 1, wherein the administrations of the composition are twice over the course of a day, under fasting conditions.

3. The method according to claim 1, wherein said dihydropyrimidine dehydrogenase inhibitor is 2,4-dihydroxy-5-chloropyridine.

4. The method according to claim 1, wherein the two administrations of the composition are separated by 6-12 hours.

5. The method according to claim 1, wherein the fasting conditions occur at least one hour before or after a meal.

6. The method according to claim 5, wherein the fasting conditions occur at least one hour before a meal.

7. The method according to claim 3, wherein the 2,4-dihydroxy-5-chloropyridine (B) and the oxonic acid or a pharmaceutically acceptable salt thereof (C) are used in a molar ratio of (B):(C)=0.4:1.

8. The method according to claim 1, wherein the pharmaceutically acceptable salt is potassium oxonate.

9. The method according to claim 3, wherein said tegafur, said 2,4-dihydroxy-5-chloropyridine, and said oxonic acid or a pharmaceutically acceptable salt thereof are used in a molar ratio of (tegafur):(2,4-dihydroxy-5-chloropyridine):(oxonic acid or a pharmaceutically acceptable salt thereof =1:0.4:1.

10. The method according to claim 1, wherein said tegafur is administered at a dose of between 20 mg/m² to 45 mg/m² twice daily.

11. The method according to claim 1, further comprising administering a chemotherapy agent selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel.

12. The method according to claim 11, wherein said chemotherapy agent is cisplatin administered intravenously.

13. The method according to claim 12, wherein said cisplatin is administered at a dose between 50-80 mg/m² daily.

14. A method for treating cancer in a mammal wherein the cancer is susceptible to 5-fluorouracil therapy, the method comprising simultaneously administering to the mammal a therapeutically effective amount of tegafur, 2,4-dihydroxy-5-chloropyridine in an amount effective for potentiating the antitumor effect of tegafur, and oxonic acid or a pharmaceutically acceptable salt thereof in an amount effective for suppressing gastrointestinal toxicity, under fasting conditions.

15. The method according to claim 14, wherein the administrations are twice over the course of a day, under fasting conditions.

16. The method according to claim 14, wherein the two administrations are separated by 6-12 hours.

17. The method according to claim 14, wherein the molar ratio of (tegafur):(2,4-dihydroxy-5-chloropyridine):(oxonic acid or a pharmaceutically acceptable salt thereof) is preferably 1:0.4:1.

18. The method according to claim 14, wherein said tegafur is administered in a dose of between 20 mg/m² to 45 mg/m² twice daily.

19. The method according to claim 14, wherein said fasting conditions occur at least one hour before or after a meal.

20. The method according to claim 14, wherein said fasting conditions occur one hour before a meal.

21. A method for inhibiting inflammation and gastrointestinal toxicity caused by the administration of tegafur, comprising administering oxonic acid or a pharmaceutically acceptable salt thereof in an amount effective for suppressing gastrointestinal toxicity, simultaneously with said tegafur to a patient in need of such treatment, wherein said tegafur and oxonic acid or a pharmaceutically acceptable salt thereof are administered under fasting conditions.

22. The method according to claim 21, wherein the administrations of the composition are twice a day under fasting conditions.

23. The method according to claim 21, further comprising simultaneously administering a dihydropyrimidine dehydrogenase inhibitor in an amount effective for potentiating the antitumor effect of tegafur.

24. A method for decreasing the breakdown of oxonic acid in the gastrointestinal tract of a patient undergoing treatment with tegafur, comprising administering oxonic acid simultaneously with said tegafur to said patient under fasting conditions.

25. The method according to claim 24, wherein the administrations of tegafur simultaneously with oxonic acid are twice daily under fasting conditions.

26. The method according to claim 24, further comprising simultaneously administering a dihydropyrimidine dehydrogenase inhibitor in an amount effective for potentiating the antitumor effect of tegafur.

27. The method according to claim 25, wherein said dihydropyrimidine dehydrogenase inhibitor is 2,4-dihydroxy-5-chloropyridine.

28. The method according to claim 25, wherein said fasting conditions occur at least one hour before or after a meal.

29. A kit for treating cancer in a mammal, comprising tegafur, 2,4-dihydroxy-5-chloropyridine, oxonic acid or a pharmaceutically acceptable salt thereof in dosages suitable for administration twice a day, and optionally comprising an anti-tumor agent selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel, the kit further comprising an indicator that reminds that said tegafur, said 2,4-dihydroxy-5-chloropyridine, and said oxonic acid or a pharmaceutically acceptable salt thereof should be administered under fasting conditions.

30. A pharmaceutical composition for potentiating the antitumor effect of tegafur while reducing gastrointestinal toxicity in a patient in need of such treatment, comprising (A) a therapeutically effective amount of tegafur, (B) a dihydropyrimidine dehydrogenase inhibitor in an amount effective for potentiating the antitumor effect and (C) oxonic acid or a pharmaceutically acceptable salt thereof in an amount effective for suppressing gastrointestinal toxicity, and optionally an additional anti-tumor agent selected from the group consisting of cisplatin, 1,3-bis(2-chloroethyl)-1-nitrosourea, docetaxel, doxorubicin, epirubicin, etoposide, methotrexate, mitomycin, gemcitabine, carboplatin, gefitinib, pemetrexed, Avastin, Cetuximab and paclitaxel, the pharmaceutical composition being contained in a package that comprises an indicator that reminds the patient that the pharmaceutical composition should be administered under fasting conditions.
